(19) United States
(12) Patent Application Publication
(10) Pub. No.: US 2017/0176472 A1
(43) Pub. Date: Jun. 22, 2017

(54) SYSTEMS AND METHODS FOR POINT-OF-CARE HDL AND LDL PARTICLE ASSAY

(71) Applicant: Polymer Technology Systems, Inc., Indianapolis, IN (US)

(72) Inventors: Aniruddha Patwardhan, Fishers, IN (US); Gary L. Hughes, Camby, IN (US)

(21) Appl. No.: 15/382,367

(22) Filed: Dec. 16, 2016

Related U.S. Application Data
(60) Provisional application No. 62/268,971, filed on Dec. 17, 2015.

(51) Int. Cl. G01N 33/92

(52) U.S. Cl. CPC G01N 33/92; G01N 2333/775; G01N 2800/32; G01N 2800/50

(57) ABSTRACT

A system for testing for LDL-Particles (LDL-P) and HDL-Particles (HDL-P) includes a first lateral flow test strip and a second lateral flow test strip; and a dosing area, the dosing area interconnected with the first and second lateral flow test strips. The system further includes a collector for collecting a sample; and a first mixer for receiving the sample from the collector, the mixer including buffers for mixing with the sample, the first mixer for dosing the sample pad, wherein the first lateral flow test strip provides for the detection of HDL-P and the second lateral flow test strip provides for the detection of LDL-P.

### CROSS REFERENCE TO RELATED APPLICATION

This application claims the benefit of U.S. Provisional Patent Application 62/268,971 filed Dec. 17, 2015, and hereby incorporated by reference to the same extent as though fully disclosed herein.

### BACKGROUND

Heart disease is a leading cause of death in today's society. The monitoring of blood analytes to assist in the monitoring and prediction of heart disease frequently is conducted by medical personnel. In many scenarios, a blood sample must be taken far in advance of meeting with medical personnel so that laboratory testing may occur. Furthermore, recent developments in research related to heart disease have determined that certain blood analytes may be more relevant to the prediction and monitoring of heart disease than others. Typically, low-density lipoprotein is known to cause cardiovascular disease by transporting cholesterol to the artery wall. Several studies have demonstrated showing the cholesterol-carrying capacity of such molecules. However, it has been determined that LDL cholesterol (LDL-C) is only one property of LDL-related particles. LDL particles (LDL-P) also affect the progression of cardiovascular disease. In fact, in some scenarios, it has been determined that the measurement of LDL-P is more instructive in determining heart disease risk than measuring LDL-C. Similarly, HDL-P is important to the measurement of heart disease risk. Therefore, it would be desirable to provide a point-of-care test that would test for LDL-P and HDL-P.

### BRIEF SUMMARY

In one embodiment, a system for testing for LDL-P and HDL-P includes a first lateral flow test strip and a second lateral flow test strip; and a dosing area, the dosing area interconnected with the first and second lateral flow test strips. The system further includes a collector for collecting a sample; and a first mixer for receiving the sample from the collector, the mixer including buffers for mixing with the sample, the first mixer for dosing the sample pad, wherein the first lateral flow test strip provides for the detection of HDL-P and the second lateral flow test strip provides for the detection of LDL-P. Optionally, the first lateral flow test strip includes a first stripe of colored particles coated with anti Apo A-1 antibody and a second stripe of Apo A antigen. In one alternative, the second lateral flow test strip includes a first stripe of colored particles coated with anti Apo B-100 antibody and a second stripe of Apo B-100 antigen. Optionally, the first and second lateral flow test strips include conjugate, nitrocellulose, and nylon membranes. In one alternative, the system further includes an optical meter for reading the first and second lateral flow test strips, the meter configured to receive the first and second lateral flow test strips.

In one embodiment, a method of testing for LDL-P and HDL-P includes providing a meter, a first lateral flow test strip; a second lateral flow test strip; a dosing area, the dosing area interconnected with the first and second lateral flow test strips; a collector for collecting a sample; and a first mixer. The method further includes collecting a sample with the collector, and mixing the sample with the mixer. The method further includes dosing the sample on the dosing area and laterally flowing the sample across the first and second lateral flow test strips. The method further includes reading the first lateral flow test strip to determine a concentration of HDL-P in the sample, and reading the second lateral flow test strip to determine a concentration of LDL-P in the sample. Optionally, the first lateral flow test strip includes a first stripe of colored particles coated with anti Apo A-1 antibody and a second stripe of Apo A antigen. In one alternative, the second lateral flow test strip includes a first stripe of colored particles coated with anti Apo B-100 antibody and a second stripe of Apo B-100 antigen. Alternatively, the first and second lateral flow test strips include conjugate, nitrocellulose, and nylon membranes. Optionally, the method further includes calibrating the meter by comparing a color produced to a known standard for a plurality of concentrations. In one alternative, the known standard is the Liposcience (now LabCorp.'s) NMR® method. In another alternative, the known standard is the Roche's “Tina-quant Apo lipoprotein B ver. 2®” assay or any on-market Apo lipoprotein B-100 assay.

In one embodiment, a system for testing for LDL-P and HDL-P includes a first lateral flow test strip and a second lateral flow test strip; and a dosing area, the dosing area interconnected with the first and second lateral flow test strips. The system further includes a collector for collecting a sample; and a first mixer for receiving the sample from the collector, the mixer including buffers for mixing with the sample, the first mixer for dosing the first and second lateral flow test strips, wherein the first lateral flow test strip provides for the detection of HDL-P and the second lateral flow test strip provides for the detection of LDL-P. Optionally, the first lateral flow test strip includes a first stripe of colored particles coated with anti Apo A-1 antibody and a second stripe of Apo A antigen. In one alternative, the second lateral flow test strip includes a first stripe of colored particles coated with anti Apo B-100 antibody and a second stripe of Apo B-100 antigen. Optionally, the first and second lateral flow test strips include conjugate, nitrocellulose, and nylon membranes. In one alternative, the system further includes an optical meter for reading the first and second lateral flow test strips, the meter configured to receive the first and second lateral flow test strips.

### BRIEF DESCRIPTION OF THE DRAWINGS

FIG. 1 shows one embodiment of the components of the lateral flow strip architecture;

FIG. 2 shows one embodiment of a device for HDL & LDL particle detection based on antibody-antigen interactions;

FIG. 3 shows one embodiment of a sampler for use with the device of FIG. 2;

FIG. 4 shows a mixer for use with the device of FIG. 2 and the sampler of FIG. 3; and

FIG. 5 shows a graph of reflectance vs. concentration of Apo lipoprotein (Apo-A or Apo-B).

### DETAILED DESCRIPTION

Certain terminology is used herein for convenience only and is not to be taken as a limitation on the embodiments of the systems and methods for point-of-care (POC) HDL and LDL particle assay. In the drawings, the same reference letters are employed for designating the same elements throughout the several figures.

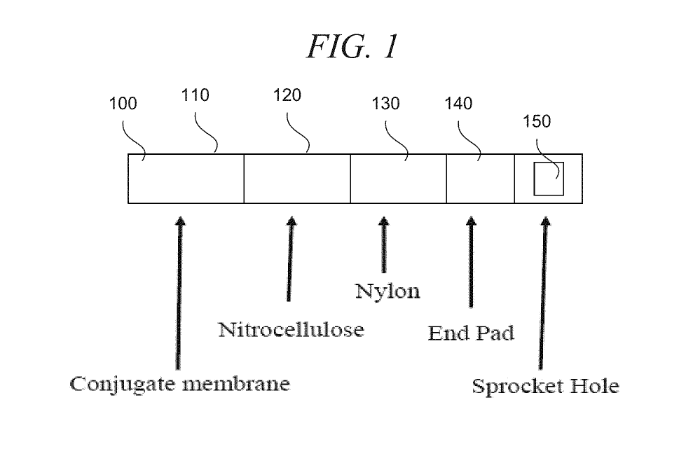

In one embodiment, the point-of-care particle assay device employs a lateral flow methodology that uses antibody-antigen interaction-based detection. In this lateral flow method, the conjugate membrane, nitrocellulose, and nylon membranes are layered in such a way as to obtain easy plasma/fluid flow which enables the analytes to be captured on the membranes in different zones as shown in FIG. 1. FIG. 1 shows one embodiment of the components of the lateral flow strip architecture. FIG. 1 shows a lateral flow test strip 100. The lateral flow test strip 100 includes a conjugate membrane 110, a nitrocellulose membrane 120, a nylon membrane 130, an end pad 140, and a sprocket hole 150 for mounting the lateral flow test strip on a test strip holder, cartridge, or cassette.

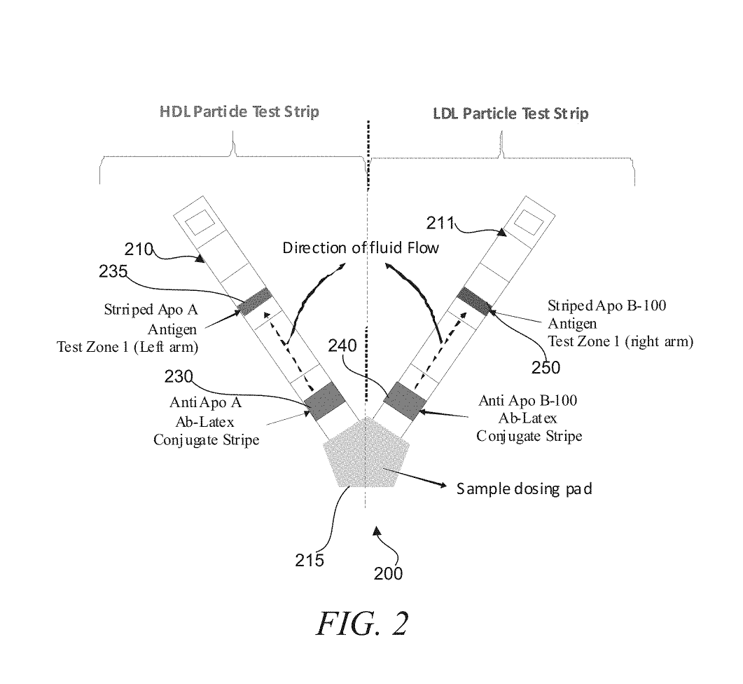

In one embodiment, a combined test strip step-up is provided as shown in FIG. 2. The device 200 also includes a sample dosing pad 215 that receives a sample and spreads it to the lateral flow test strips.

The HDL-P arm 210, a lateral flow method using an antibody-antigen interaction to quantify the levels of HDL's Apo A protein in the sample, is utilized. An antibody-antigen stripe 230 (anti Apo A-1 Ab-latex conjugate stripe) is included in the lateral flow strip. Blue particles (or alternatively other detectable particle colors) coated with anti Apo A-1 antibody will be striped as shown in FIG. 2 at stripe 230. The Apo A antigen will be striped in zone one at stripe 235.

The LDL-P arm 211, a lateral flow method using an antibody-antigen interaction to quantify the levels of HDL's Apo B-100 protein in the sample, is utilized. An antibody-antigen stripe 240 (anti Apo B Ab-latex conjugate stripe) is included in the lateral flow strip. Blue particles (or alternatively other detectable particle colors) coated with anti Apo B-100 antibody will be striped as shown in FIG. 2 at stripe 240. The Apo B-100 antigen will be striped in zone one at stripe 250.

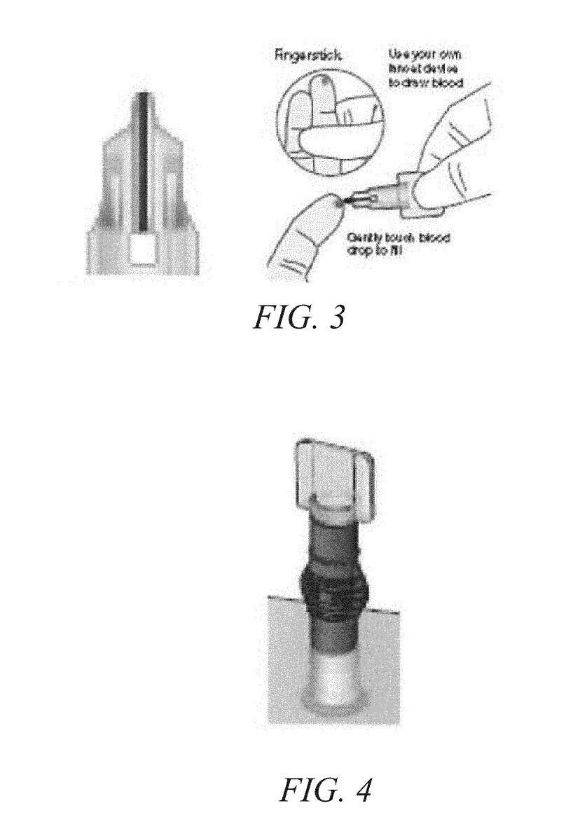

Dosing and quantification occur according to a number of steps. FIG. 3 shows a sample collector with whole blood and the method for collecting capillary whole blood from a finger stick. FIG. 4 shows a sample collector with a mixer containing reagents containing proprietary buffer formulary (containing salt and enzymes). The sampler includes a one-stop snap cap with a perforator for perforating the chamber containing water which may later be mixed with the chamber and then dosed by removing the cap.

A venous or capillary sample will be collected by the collector as shown in FIG. 3, mixed with proprietary buffers by sample mixers (as shown in FIG. 4), and dosed on the sample pad. After dosing, the solution will wick on both arms evenly. The following physical phenomenon will ensue.

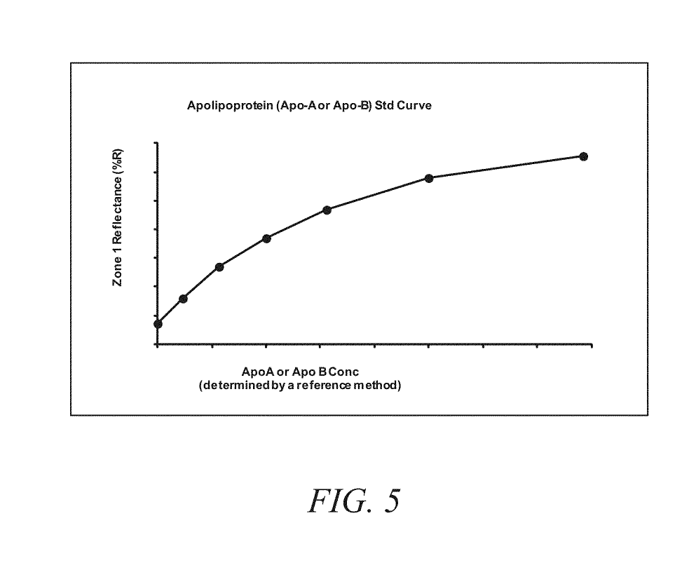

The blue particles in stripes 230, 240 in the arms 210, 211 of the device will interact with the sample and migrate along the length of the lateral flow strip. The blue particles coated with anti Apo B-100 antibody will be captured in the test zone one at stripe 250 (which is striped with Apo B-100 antigen-protein conjugate) when a very small amount of LDL-P is present in the sample. If a large amount of LDL-P is present in the sample, the LDL will stick to the blue particles resulting in proportionally lower capture of the blue particles in zone one at stripe 250. In this particular immunochemistry method, a direct relationship exists between the analyte concentrations to the light reflected from the “capture” in zone one at stripe 250 (for both arms of the device) as shown in the dose response, FIG. 5. A lower reflectance reading will yield a lower value, while a high reflectance reading will yield a higher analyte concentration. In embodiments, an LDL-P is calibrated (FIG. 5) against the Liposcience (now LabCorp.'s) NMR® method to yield a particle count or concentration of LDL particles in the same. The Roche's “Tina-quant Apo lipoprotein B-100 ver. 2®” assay or any on-market Apo lipoprotein B-100 assay may be used for calibration, thus enabling a POC device now to report the Apo B-100 concentrations for the LDL lipoproteins.

The blue particle coated with anti Apo A-1 antibody will be captured in the test zone one at stripe 230 (which is striped with Apo A-1 antigen-protein conjugate) when a very small amount of HDL-P is present in the sample. If a large amount of HDL-P is present in the sample, the HDL will stick to the blue particles resulting in proportionally lower capture of the blue particles in zone one at stripe 235. In this particular immunochemistry method, a direct relationship exists between the analyte concentrations to the light reflected from the “capture” in zone one at stripe 235 (for both arms of the device) as shown in the dose response, FIG. 5. A lower reflectance reading will yield a lower value, while a high reflectance reading will yield a higher analyte concentration. In embodiments, an HDL-P can be calibrated (FIG. 5) against the Liposcience (now LabCorp.'s) NMR® method to report the HDL particle count or concentration. The Roche's “Tina-quant Apo lipoprotein A-1 ver. 2®” or any on-market assay for Apo lipoprotein A-1 assay may be used for calibration, thus enabling a POC device to now report the Apo A-1 concentration in the HDL lipoproteins.

Embodiments of the systems and methods described herein are constructed to determine the number of particles for high density lipoprotein (abbreviated as HDL-P) and low density lipoprotein (LDL-P) in a sample using a simple reflectance-based method, in lieu of their concentration, abbreviated as HDL-C & LDL-C. This system is advantageous because:
1. This method will yield a direct HDL- and LDL-Particle concentration, instead of the cholesterol concentration in their respective lipoprotein fraction.
2. It will be the first POC device that can give particle concentration (count) for both Apo lipoprotein A-1 and B-100.
3. It will be the first POC device that can give the concentration of Apo B-100 and Apo A-1 lipoproteins.
3. In recent literature, it has been recognized that particle concentration of HDL and LDL are of greater diagnostic value than the cholesterol concentrations.

Systems and methods for a point-of-care test (POCT) device for determining the concentration of Apo lipoprotein A in HDL particles and Apo lipoprotein B-100 in LDL particles are disclosed herein. In many embodiments, the POCT device employs a lateral flow methodology. This method is an antibody-antigen interaction to quantify the levels of analyte in the sample. In lateral flow test strips, the conjugate membrane, nitrocellulose, and nylon membranes are layered in such a way as to obtain easy plasma/fluid flow which enables the analytes to be captured on the membranes in different zones.

The samples (capillary or venous whole blood) will be collected by a sample collector as shown in FIG. 3 and placed in a mixer (redwood) as shown in FIG. 4. The mixer will contain proprietary buffers and a stabilizer. This mixture then will be dosed on the sample pad and wicked through the lateral flow membranes to capture the LDL-P and HDL-P (particles) in their respective zones as shown in FIG. 2.

In this particular immunochemistry method, a direct relationship exists between the analyte concentrations to the light reflected from the “capture” in zone 1 (for both arms of the device) as shown in the dose response of FIG. 5. A lower reflectance reading will yield a lower value, while a higher reflectance reading will yield a higher analyte concentration.

The dose response of the number of Apo lipoproteins bound is quantified by calibrating against the FDA-cleared apolioprotein assay. For example, in some embodiments, the LDL-P (particle) is calibrated against the Liposcience (now LabCorp.'s) NMR® method or via the Roche's “Tina-quant Apo lipoprotein B ver. 2®” assay or any on-market assays quantifying Apo B-100 concentrations. Similarly the HDL-P (particle) dose response is calibrated against the Liposcience NMR® method, thus enabling a POC device to now report the particle count for the HDL and the LDL lipoproteins.

In many embodiments, a premix step may be included. Typically, a sample is exposed to a premix step with a buffer solution (referred to as the sample treatment buffer). After a premix, the sample and buffer solution are applied to a test strip. The test strip includes an antibody-microparticle zone and a capture zone for capturing antibodies that have not reacted with the sample. Typically, after the lateral flow of the sample, the flow of the microparticles is measured using an optical meter.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof.

What is claimed as new and desired to be protected by Letters Patent of the United States is:

1. A system for testing for LDL-P and HDL-P, the system comprising:
a first lateral flow test strip and a second lateral flow test strip;
a dosing area, the dosing area interconnected with the first and second lateral flow test strips;
a collector for collecting a sample; and
a first mixer for receiving the sample from the collector, the mixer including buffers for mixing with the sample, the first mixer for dosing the sample pad, wherein the first lateral flow test strip provides for the detection of HDL-P and the second lateral flow test strip provides for the detection of LDL-P.

2. The system of claim 1, wherein the first lateral flow test strip includes a first stripe of colored particles coated with anti Apo A-1 antibody and a second stripe of Apo A antigen.

3. The system of claim 1, wherein the second lateral flow test strip includes a first stripe of colored particles coated with anti Apo B-100 antibody and a second stripe of Apo B-100 antigen.

4. The system of claim 1, wherein the first and second lateral flow test strips include conjugate, nitrocellulose, and nylon membranes.

5. The system of claim 1, further comprising an optical meter for reading the first and second lateral flow test strips, the meter configured to receive the first and second lateral flow test strips.

6. A method of testing for LDL-P and HDL-P, the method comprising:
providing a meter; a first lateral flow test strip; a second lateral flow test strip; a dosing area, the dosing area interconnected with the first and second lateral flow test strips; a collector for collecting a sample; and a first mixer;
collecting a sample with the collector;
mixing the sample with the mixer;
dosing the sample on the dosing area;
laterally flowing the sample across the first and second lateral flow test strips;
reading the first lateral flow test strip to determine a concentration of HDL-P in the sample; and
reading the second lateral flow test strip to determine a concentration of LDL-P in the sample.

7. The method of claim 6, wherein the first lateral flow test strip includes a first stripe of colored particles coated with anti Apo A-1 antibody and a second stripe of Apo A antigen.

8. The method of claim 6, wherein the second lateral flow test strip includes a first stripe of colored particles coated with anti Apo B-100 antibody and a second stripe of Apo B-100 antigen.

9. The method of claim 6, wherein the first and second lateral flow test strips include conjugate, nitrocellulose, and nylon membranes.

10. The method of claim 6, further comprising calibrating the meter by comparing a color produced to a known standard for a plurality of concentrations.

11. The method of claim 10, wherein the known standard is the Liposcience (LabCorp.'s) NMR® method.

12. The method of claim 10, wherein the known standard is the Roche's “Tina-quant Apo lipoprotein A-1 for HDL and B-100 for LDL ver. 2®” assay.

13. A system for testing for LDL-P and HDL-P, the system comprising:
a first lateral flow test strip and a second lateral flow test strip;
a collector for collecting a sample; and
a first mixer for receiving the sample from the collector, the mixer including buffers for mixing with the sample, the first mixer for dosing the first and second lateral flow test strips, wherein the first lateral flow test strip provides for the detection of HDL-P and the second lateral flow test strip provides for the detection of LDL-P.

14. The system of claim 13, wherein the first lateral flow test strip includes a first stripe of colored particles coated with anti Apo A-1 antibody and a second stripe of Apo A antigen.

15. The system of claim 13, wherein the second lateral flow test strip includes a first stripe of colored particles coated with anti Apo B-100 antibody and a second stripe of Apo B-100 antigen.

16. The system of claim 13, wherein the first and second lateral flow test strips include conjugate, nitrocellulose, and nylon membranes.

17. The system of claim 13, further comprising an optical meter for reading the first and second lateral flow test strips, the meter configured to receive the first and second lateral flow test strips.
